(19) United States
(12) Patent Application Publication
(10) Pub. No.: US 2026/0041918 A1
(43) Pub. Date: Feb. 12, 2026

(54) NEUROMODULATION PROGRAMMING USING COMBINED MODULATION CONFIGURATIONS

(71) Applicant: Boston Scientific Neuromodulation Corporation, Valencia, CA (US)

(72) Inventors: Lisa Denise Moore, Glendale, CA (US); Mahsa Malekmohammadi, Sherman Oaks, CA (US); Richard Mustakos, Simi Valley, CA (US); G. Karl Steinke, Valencia, CA (US)

(21) Appl. No.: 19/294,645

(22) Filed: Aug. 8, 2025

Related U.S. Application Data
(60) Provisional application No. 63/681,675, filed on Aug. 9, 2024.

(51) Int. Cl. A61N 1/36; A61N 1/02; A61N 1/05

(52) U.S. Cl. CPC A61N 1/36185; A61N 1/025; A61N 1/0551; A61N 1/36167

(57) ABSTRACT

A system may include a neurostimulator and a processing system, where the neuromodulator includes a directional lead. The processing system may be configured to perform a process that includes determining at least one stimulation field model (SFM) from at least a first vector that extends from a first virtual electrode on the directional lead and a second vector that extends from a second virtual electrode on the directional lead and determining a first modulation configuration corresponding to the first vector from the first virtual electrode and determining a second modulation configuration corresponding to the second vector from the second virtual electrode. The process may further include combining the first modulation configuration and the second modulation configuration into a combined modulation configuration and delivering neuromodulation via a single timing channel using the combined modulation configuration.

### CLAIM OF PRIORITY

This application claims the benefit of U.S. Provisional Application No. 63/681,675, filed on Aug. 9, 2024, which is hereby incorporated by reference in its entirety.

### TECHNICAL FIELD

This document relates generally to medical devices, and more particularly, to systems, devices and methods programming a neuromodulation system.

### BACKGROUND

Medical devices may include therapy-delivery devices configured to deliver a therapy to a patient and/or monitors configured to monitor a patient condition via user input and/or sensor(s). Examples include wearable devices such as but not limited to, transcutaneous electrical neural stimulators (TENS), external or implantable stimulation devices such as but not limited to spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, and the like.

A therapy device may be configured or programmed to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. Settings of the therapy device, including stimulation parameters, may be programmed based on observed clinical effects so that the therapy provides desirable intended effects (e.g., reduced tremor, bradykinesia, and dyskinesia for a PD therapy, desirable pain relief or paresthesia coverage for a pain therapy) while avoiding undesirable side effects.

Current clinical support tools use algorithms that target stimulation to a single region on each lead. Even though stimulation of different regions along the same lead could improve symptoms or counteract side effects, searching for a secondary target on the same lead may be difficult using those algorithms.

It is desirable to provide improved systems for finding and creating combinations of programs for two or more distinct stimulation targets.

### SUMMARY

An example (e.g., “Example 1”) of a system may include a neurostimulator and a processing system, where the neurostimulator includes a plurality of leads. The processing system may be configured to perform a process that includes determining a first fractionalization to stimulate a first target using the neurostimulator, determining a second fractionalization to stimulate a second target using the neurostimulator, and determining a combined fractionalization, using the first fractionalization and the second fractionalization, for stimulating both the first target and the second target using a single timing channel in the neurostimulator. The neurostimulator is configured to stimulate the first target and the second target using the single timing channel and the combined fractionalization.

In Example 2, the subject matter of Example 1 may optionally be configured such that the first fractionalization is part of a first stimulation parameter set configured for use by the neurostimulator to stimulate the first target, the second fractionalization is part of a second stimulation parameter set configured for use by the neurostimulator to stimulate the second target, the first stimulation parameter set and the second stimulation parameter set have a same pulse width and a same frequency, and a combined parameter set configured for use by the neurostimulator to simultaneously stimulate both the first target and the second target include the combined fractionalization, the same pulse width and the same frequency.

In Example 3, the subject matter of any one or more of Examples 1-2 may optionally be configured such that the combined fractionalization is determined by weighting the first fractionalization and the second fractionalization.

In Example 4, the subject matter of Example 3 may optionally be configured such that the processing system is configured to determine a first area amplitude for stimulating the first target and determine a second area amplitude for stimulating the second target. The first area amplitude is distributed among a first set of active electrodes according to the first fractionalization and the second area amplitude is distributed among a second set of active electrodes according to the second fractionalization. A combined amplitude may be determined by summing the first area amplitude and the second area amplitude. The combined fractionalization is determined by amplitude weighting a fractional contribution for the first set of active electrodes using the first area amplitude and dividing by the combined amplitude and by amplitude weighting a fractional contribution for the second set of active electrodes using the second area amplitude and dividing by the combined amplitude. The neurostimulator is configured to use the combined amplitude and the combined fractionalization to stimulate the first target and the second target.

In Example 5, the subject matter of any one or more of Examples 1-4 may optionally be configured such that the neurostimulator includes at least one lead, the plurality of electrodes are on the at least one lead, and the at least one lead includes a directional lead or a linear lead.

In Example 6, the subject matter of any one or more of Examples 1-5 may optionally be configured such that stimulation of the first target addresses a first symptom and stimulation of the second target addresses a second symptom.

In Example 7, the subject matter of any one or more of Examples 1-6 may optionally be configured such that the processing system includes a programming tool configured to both determine the first fractionalization to stimulate the first target and determine the second fractionalization to stimulate the second target. In some examples, the programming tool may be able to receive user input on stimulation location (vector orientation) and size (amplitude) to define one or both targets. The combined fractionalization for a single timing channel may be steered.

In Example 8, the subject matter of Example 7 may optionally be configured such that the programming tool is configured to determine neurostimulation target information indicative of at least one of the first target and the second target by receiving the neurostimulation target information via a user interface, receiving medical imaging data, receiving a sensor signal indicative of a sensed parameter, or receiving user inputs regarding at least one a clinical effect or side effect of the neurostimulation.

In Example 9, the subject matter of any one or more of Examples 7-8 may optionally be configured such that the programming tool is configured to determine neurostimulation target information indicative of at least one of the first target and the second target using at least one of anatomical information indicative of an anatomical structure, electrophysical data, or neuromodulation response information. The neuromodulation response information may include at least one of a heat map indicative of a desired response and/or undesired response to neuromodulation sites, sensor feedback when neuromodulation is delivered at neuromodulation sites, or user feedback indicative of symptom relief and/or experienced side effects when the neuromodulation is delivered at the neuromodulation sites.

In Example 10, the subject matter of any one or more of Examples 1-9 may optionally be configured such that the process performed by the processing system further includes determining a third fractionalization to stimulate a third target, and the combined fractionalization is determined to simultaneously stimulate the first target, the second target and the third target using the single timing channel. The combined fractionalization may be determined using the first fractionalization, the second fractionalization, and the third fractionalization.

In Example 11, the subject matter of Example 10 may optionally be configured such that the process performed by the processing system includes combining fractionalizations using different combinations of the first fractionalization, the second fractionalization and the third fractionalization to create a plurality of combined solutions, and comparing the combined solutions to determine best fractionalization to stimulate all targets.

In Example 12, the subject matter of any one or more of Examples 1-11 may optionally be configured such that the processing system is configured to determine a first stimulation field to stimulate the first target, determine the first fractionalization based on the first stimulation field, determine a second stimulation field to stimulate the second target, and determine the second fractionalization based on the second stimulation field.

In Example 13, the subject matter of any one or more of Examples 1-12 may optionally be configured such that the processing system is configured to receive a first target input indicative of the first target and a second target input indicative of the second target.

In Example 14, the subject matter of Example 13 may optionally be configured such that the processing system is configured to receive a first avoidance input indicative of a first avoidance region and a second avoidance input indicative of a second avoidance region.

In Example 15, the subject matter of any one or more of Examples 1-14 may optionally be configured such that the first stimulation field corresponds to a first stimulation field model (SFM) that is based on the first fractionalization; and the second stimulation field corresponds to a second stimulation field model (SFM) that is based on the second fractionalization.

Example 16 includes subject matter (such as a method, means for performing acts, machine readable medium including instructions that when performed by a machine cause the machine to perform acts, or an apparatus to perform). The subject matter may include determining a first fractionalization to stimulate a first target using a neurostimulator that includes a plurality of electrodes, determining a second fractionalization to stimulate a second target using the neurostimulator, determining a combined fractionalization, using the first fractionalization and the second fractionalization, for stimulating both the first target and the second target using a single timing channel in the neurostimulator, and stimulating the first target and the second target using the single timing channel in the neurostimulator and the combined fractionalization.

In Example 17, the subject matter of Example 16 may optionally be configured such that the first fractionalization is part of a first stimulation parameter set configured for use by the neurostimulator to stimulate the first target, the second fractionalization is part of a second stimulation parameter set configured for use by the neurostimulator to stimulate the second target, the first stimulation parameter set and the second stimulation parameter set have a same pulse width and a same frequency, and the first target and the second target are simultaneously stimulated using a combined parameter set that includes the combined fractionalization, the same pulse width and the same frequency.

In Example 18, the subject matter of any one or more of Examples 16-17 may optionally be configured such that the combined fractionalization is determined by weighting the first fractionalization and the second fractionalization.

In Example 19, the subject matter of any one or more of Examples 16-18 may optionally be configured to further include determining a first area amplitude for stimulating the first target and distributing the first area amplitude among a first set of active electrodes according to the first fractionalization, determining a second area amplitude for stimulating the second target and distributing the second area amplitude among a second set of active electrodes according to the second fractionalization, and determining a combined amplitude by summing the first area amplitude and the second area amplitude. The combined fractionalization may be determined by amplitude weighting a fractional contribution for the first set of active electrodes using the first area amplitude and dividing by the combined amplitude and by amplitude weighting a fractional contribution for the second set of active electrodes using the second area amplitude and dividing by the combined amplitude. The combined amplitude and the combined fractionalization may be used to stimulate the first target and the second target using the single timing channel in the neurostimulator.

In Example 20, the subject matter of any one or more of Examples 16-19 may optionally be configured such that the neurostimulator includes at least one lead, the plurality of electrodes are on the at least one lead, and the at least one lead includes a directional lead or a linear lead.

In Example 21, the subject matter of any one or more of Examples 16-20 may optionally be configured such that stimulation of the first target addresses a first clinical benefit or side effect and stimulation of the second target addresses a second clinical benefit or side effect.

In Example 22, the subject matter of any one or more of Examples 16-21 may optionally be configured to further include using a programming tool to both determine the first fractionalization to stimulate the first target and determine the second fractionalization to stimulate the second target. In some examples, the programming tool may be able to receive user input on stimulation location (vector orientation) and size (amplitude) to define one or both targets. The combined fractionalization for a single timing channel may be steered.

In Example 23, the subject matter of Example 22 may optionally be configured to further include using the programming tool to determine neurostimulation target information indicative of at least one of the first target and the second target by: receiving the neurostimulation target information via a user interface; receiving medical imaging data; receiving a sensor signal indicative of a sensed parameter; or receiving user inputs regarding at least one a clinical effect or side effect of the neurostimulation.

In Example 24, the subject matter of any one or more of Examples 22-23 may optionally be configured to further include using the programming tool to determine neurostimulation target information indicative of at least one of the first target and the second target using at least one of anatomical information indicative of an anatomical structure, electrophysical data, or neuromodulation response information, wherein the neuromodulation response information includes at least one of a heat map indicative of a desired response and/or undesired response to neuromodulation sites, sensor feedback when neuromodulation is delivered at neuromodulation sites, or user feedback indicative of symptom relief and/or experienced side effects when the neuromodulation is delivered at the neuromodulation sites.

In Example 25, the subject matter of any one or more of Examples 16-24 may optionally be configured to further include determining a third fractionalization to stimulate a third target, wherein the combined fractionalization is determined to simultaneously stimulate the first target, the second target and the third target using the single timing channel, the combined fractionalization is determined using the first fractionalization, the second fractionalization, and the third fractionalization.

In Example 26, the subject matter of Example 25 may optionally be configured to further include combining fractionalizations using different combinations of the first fractionalization, the second fractionalization and the third fractionalization to create a plurality of combined solutions and comparing the combined solutions to determine best fractionalization to stimulate all targets.

In Example 27, the subject matter of any one or more of Examples 16-26 may optionally be configured to further include determining a first stimulation field to stimulate the first target, determining the first fractionalization based on the first stimulation field, determining a second stimulation field to stimulate the second target, and determining the second fractionalization based on the second stimulation field.

In Example 28, the subject matter of any one or more of Examples 16-27 may optionally be configured to further include receiving a first target input indicative of the first target and a second target input indicative of the second target.

In Example 29, the subject matter of Example 28 may optionally be configured to further include receiving a first avoidance input indicative of a first avoidance region and a second avoidance input indicative of a second avoidance region.

In Example 30, the subject matter of any one or more of Examples 16-29 may optionally be configured such that the first stimulation field corresponds to a first stimulation field model (SFM) that is based on the first fractionalization, and the second stimulation field corresponds to a second stimulation field model (SFM) that is based on the second fractionalization.

Example 31 includes subject matter that includes non-transitory machine-readable medium including instructions, which when executed by a machine, cause the machine to perform a method. The method may include, by way of example and not limitation, any of the subject matter for one or more of Examples 16-30. The machine-readable medium may include instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine-readable medium” is intended to include at least one machine-readable medium (e.g., two or more media which may be of the same type of media (such as but not limited to different nonvolatile semiconductor memory arrays) or different type of media (such as but not limited to a hard disk and a non-volatile semiconductor memory array). Furthermore, the term “machine” may include at least one processor, including one processor to implement all of the instructions, at least two processors where one processor operates on some of the instructions and other processor(s) operate on other instructions, or at least two processors where each processor is capable of operating on the same instructions. Thus, for example, distributed systems or systems with shared resources are contemplated.

This Summary is an overview of some of the teachings of the present application and not intended to be an exclusive or exhaustive treatment of the present subject matter.

Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates an example of an electrical stimulation system that may be used to deliver deep brain stimulation (DBS).

FIG. 2 illustrates an example of an implantable pulse generator (IPG) that may be used in a DBS system.

FIGS. 3A-3B illustrate examples of leads that may be coupled to an IPG to deliver electrostimulation such as DBS.

FIG. 4 illustrates an example of a computing device for programming or controlling the operation of an electrostimulation system.

FIG. 5 illustrates, by way of example and not limitation, the electrical therapy-delivery system.

FIG. 6 illustrates, by way of example and not limitation, a stimulation parameter control system and a part of the environment in which it may operate.

FIG. 7 illustrates a therapy being delivered according to a parameter set.

FIG. 8 illustrates a therapy space, which includes different parameter sets potentially available for delivering the therapy.

FIG. 9 illustrates, by way of example and not limitation, an example of a directional lead and a representation of a grid that illustrates steering of a virtual electrode with respect to physical electrodes on the directional lead.

FIG. 10 illustrates, by way of example and not limitation, two vectors extending away from a directional lead.

FIG. 11 illustrates, by way of example and not limitation, a virtual electrode steering grid for the vectors illustrated in FIG. 10.

FIG. 12 illustrates, by way of example and not limitation, a map illustrating good regions for modulation, side effect regions, and regions that have some benefit and no side effects, and also illustrating stimulation field models (SFMs) for two vectors from two virtual electrodes (VEs).

FIGS. 13A-13D illustrate, by way of example, a first configuration for providing a first vector to modulate a first target, a second configuration for providing a second vector to modulate a second target, and a combination configuration for providing both the first vector to modulate the first target and the second vector to modulate the second vector.

FIG. 14 illustrates, by way of example and not limitation, a method for creating a stimulation configuration to stimulate two or more targets.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments described herein independently determine solutions for stimulating each of two or more targets using a neurostimulator with a plurality of electrodes. The independently-determined solutions include fractionalizations for each of the targets. A combined fractionalization may be determined using the fractionalizations for each of the independently-determined solutions and the neurostimulator may use the combined fractionalization to simultaneously stimulate the two or more targets using a single timing channel.

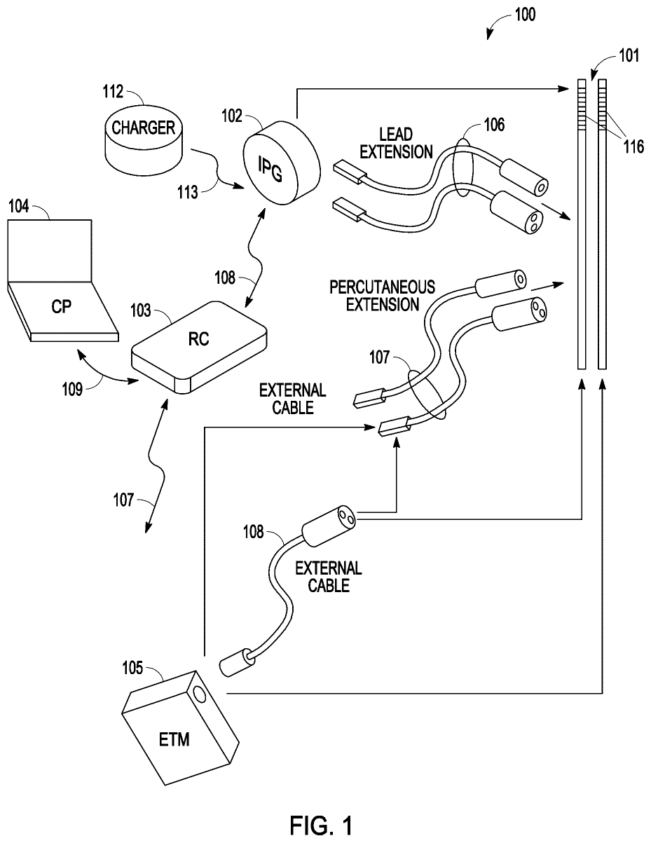

FIG. 1 illustrates, by way of example and not limitation, an electrical stimulation system 100, which may be used to deliver DBS. The electrical stimulation system 100 may generally include a one or more (illustrated as two) of implantable neurostimulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neurostimulation lead(s) 101, which carry a plurality of electrodes 116. The electrodes, when implanted in a patient, form an electrode arrangement. As illustrated, the neurostimulation leads 101 may be percutaneous leads with the electrodes arranged in-line along the neurostimulation leads or about a circumference of the neurostimulation leads. Any suitable number of neurostimulation leads can be provided. The IPG 102 includes pulse generation circuitry that delivers electrical stimulation energy in the form of a pulsed electrical waveform (i.e., a temporal series of electrical pulses) to the electrodes in accordance with a set of stimulation parameters.

The ETM 105 may also be physically connected via the percutaneous lead extensions 107 and external cable 108 to the neurostimulation lead(s) 101. The ETM 105 may have similar pulse generation circuitry as the IPG 102 to deliver electrical stimulation energy to the electrodes in accordance with a set of stimulation parameters. A programming process may be used to test different parameter sets. The ETM 105 is a non-implantable device that may be used on a trial basis after the neurostimulation leads 101 have been implanted and prior to implantation of the IPG 102, to test the responsiveness of the stimulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different stimulation parameter sets. The IPG 102 may also be operated to modify the programmed stimulation parameters to actively control the characteristics of the electrical stimulation energy output by the IPG 102. A clinician may use the CP 104 to program stimulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed stimulation parameters provided by the CP 104 may also be used to program the RC 103, so that the stimulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical stimulation generated by the IPG 102 to allow the desired parameters to be determined based on patient feedback or other feedback and for subsequently programming the IPG 102 with the desired stimulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the neurostimulation leads, and select and program the IPG with stimulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

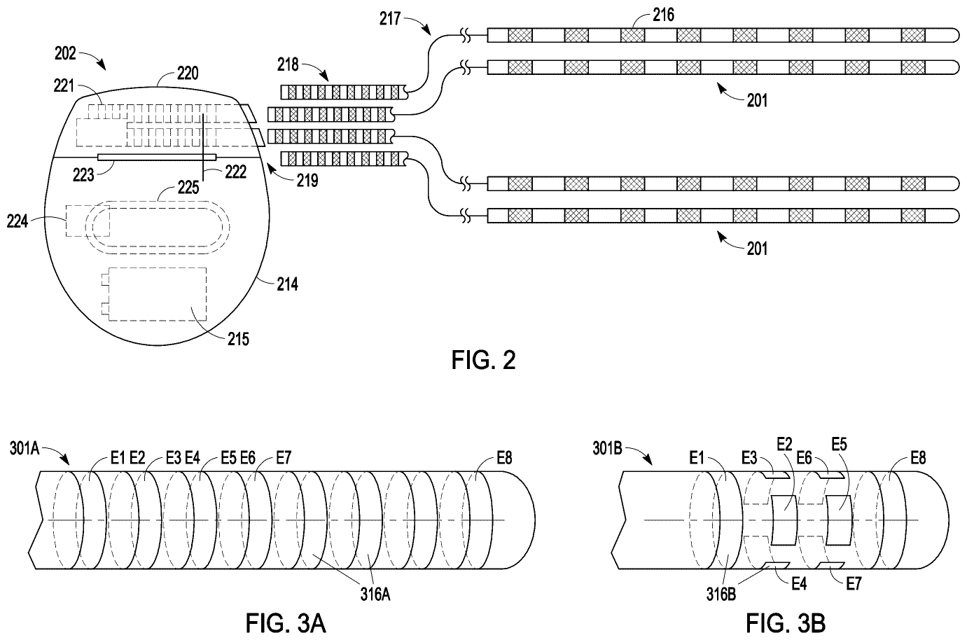

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be directional so that stimulation may be aligned with the target neurons after the neurons have been located such as based on the recorded signals. The electrodes 216 may include one or more ring electrodes, and/or one or more sets of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a Radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, MICS, and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes may either be used (an active electrode) or unused (OFF). When the electrode is used, the electrode may be used as an anode or cathode and carry anodic or cathodic current. The anodic energy contributions may be distributed across more than one anode and the cathodic energy contributions may be distributed across more than one cathode (e.g., electrode fractionalization). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead.

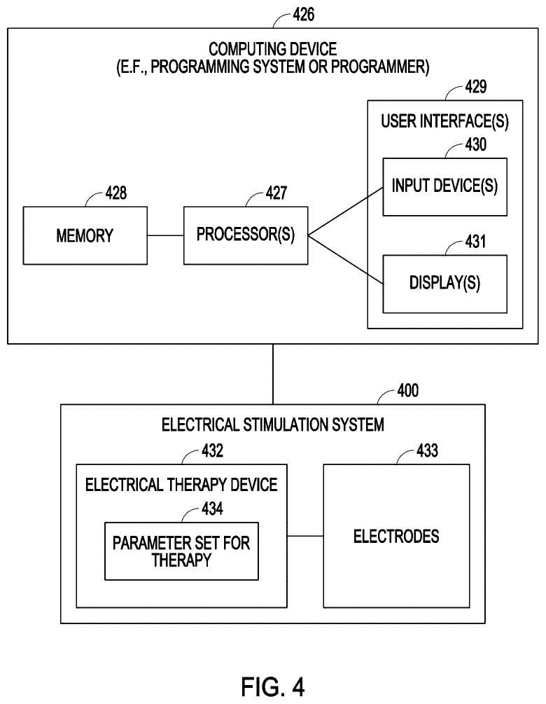

FIG. 4 illustrates, by way of example and not limitation, a computing device 426 for programming or controlling the operation of an electrical stimulation system 400. The computing device 426 may include a processor 427, a memory 428, and user interface(s) 429, which may include input device(s) 430 and display(s) 431. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1.

The computing device 426, also referred to as a programming system mor programmer, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. In some examples, the computing device 426 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the axis. CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, the closed-loop algorithm for determining or updating stimulation parameters may be implemented in a mobile device, such as a smartphone, which is connected to the IPG or an evaluating device (e.g., a wristband or watch). These devices may also record and send information to the clinician.

The processor 427 may include one or more processors that may be local to the user or non-local to the user or other components of the computing device 426. In an example, the processor 427 may execute instructions (e.g., stored in the memory 428) to determine a search space of electrode configurations and parameter values, and identify or update one or more stimulation settings that are selectable for use in electrostimulation therapies such as DBS. The search space may include a collection of available electrodes, possible electrode configurations, and possible values or value ranges of one or more stimulation parameters that may be applied to selected electrodes to deliver electrostimulation. The search space may be specific to a particular lead or a type of lead with respect to a specific neural target. As a result, for different leads or types of lead and/or for different neural targets, the processor 427 may determine respective different search spaces. A modulation configuration, which may be referred to as a stimulation setting, includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, and like.

The processor 427 may identify or modify a stimulation setting from the search space through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings, and/or instructions for implementing a testing process for testing stimulation parameters. The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

The input device(s) 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries. The display(s) 431 may be any suitable display or presentation device, such as a monitor, screen, display, or the like, and may include a printer. The display(s) 431 may be a part of a user interface configured to display information about stimulation settings (e.g., electrode configurations and stimulation parameter values and value ranges) and user control elements for programming a stimulation setting into an IPG.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

The electrical stimulation system 400 may include an electrical therapy device 432 configured to deliver an electrical therapy to electrodes 433 to treat a condition in accordance with a programmed parameter set 434 for the therapy. The illustrated electrical stimulation system 400 may be a DBS system. In some embodiments, the illustrated electrical stimulation system 400 may be an SCS system to treat pain using directional lead(s).

A modulation configuration, which may be referred to as a stimulation setting (e.g., parameter set), includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. By way of example and not limitation, the stimulation parameters may include current amplitude values, current fractionalization across electrodes, stimulation frequency, stimulation pulse width, and the like. Electrodes are capable of being selectively programmed to act as anodes (positive), cathodes (negative), or left off (zero). The electrical therapy device 432 may be configured to control or vary other parameters including, but are not limited to, the amplitude, pulse width, rate (or frequency), ON/OFF timing, and the like. Each electrode configuration, along with the electrical pulse parameters, can be referred to as a “neuromodulation parameter set.” Each set of neuromodulation parameters, including fractionalized current distribution to the electrodes (as percentage cathodic current, percentage anodic current, or off), may be stored and combined into a neuromodulation program that can then be used to modulate multiple regions within the patient.

The electrodes 433 may be distributed in an electrode arrangement using one or more leads. The electrical therapy device 432 may include a plurality of independent sources such as independent current sources for each electrode. The electrical therapy device 432 may be configured as a multi-channel (such as but not limited to four channels) system capable of simultaneously and independently generating and delivering separate stimulation waveforms to different electrode combinations. The channels may be referred to as timing channels.

Some embodiments of the electrical therapy device 432 may include electrical sensing circuitry configured to sense electrical activity (e.g., local field potentials, evoked compound actions potentials, evoked resonant neural activity (ERNA), electrospinogram, or other electrical signals) using at least some of the electrodes. Some embodiments of the neuromodulation device may include other sensor(s) that may be used to control the neuromodulation or provide context for the therapy or other events, or to detect events.

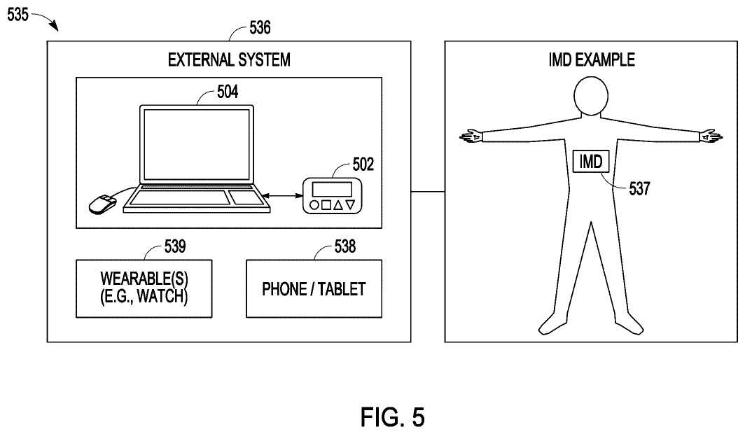

FIG. 5 illustrates, by way of example and not limitation, the electrical therapy-delivery system. The illustrated system 535 includes an external system 536 that may include at least one programming device or programming system such as computing device 426 in FIG. 4. The illustrated external system 536 may include a clinician programmer 504, similar to CP 104 in FIG. 1, configured for use by a clinician to communicate with and program the neuromodulator, and a remote control device 503, similar to RC 103 in FIG. 1, configured for use by the patient to communicate with and program the neuromodulator. For example, the remote control device 503 may allow the patient to turn a therapy on and off and/or may allow the patient to adjust patient-programmable parameter(s) of the plurality of stimulation parameters. FIG. 5 illustrates an IMD 537, although the therapy device may be an external device such as a wearable device. The external system 536 may include a network of computers, including computer(s) remotely located from the IMD 537 that are capable of communicating via one or more communication networks with the programmer 504 and/or the remote control device 503. The remotely located computer(s) and the IMD 537 may be configured to communicate with each other via another external device such as the programmer 504 or the remote control device 503. The remote control device 503 and/or the programmer 504 may allow a user (e.g., patient and/or clinician or rep) to answer questions as part of a data collection process. The external system 536 may include personal devices such as a phone or tablet 538, wearables such as a watch 539, sensors or therapy-applying devices. The watch may include sensor(s), such as sensor(s) for detecting activity, motion and/or posture. The phone and/or tablet may include camera(s), microphone(s), accelerometer(s) or other sensors that can be used to provide feedback. Other wearable sensor(s) may be configured for use to detect activity, motion and/or posture of the patient.

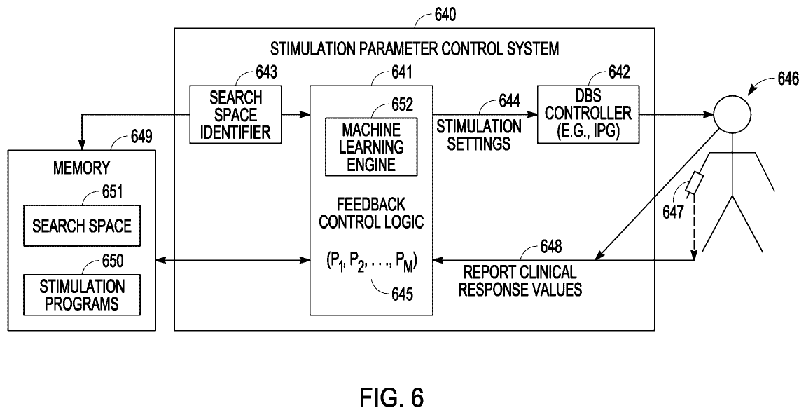

FIG. 6 illustrates, by way of example and not limitation, a stimulation parameter control system and a part of the environment in which it may operate. The stimulation parameter control system 640, which may be implemented as a part of the processor 427 in FIG. 4, may include a feedback control logic 641, a DBS controller 642, and a search space identifier 643. DBS is used as an example. It is noted that the system may be implemented for other stimulation therapies such as, but not limited to, SCS or PNS. The feedback control logic 641 may be implemented in, for example, the CP 104 or the RC 103 in FIG. 1. The feedback control logic 641 may determine or modify one or more stimulation settings 644 for a stimulation lead at a target stimulation region, such as a region in a brain hemisphere. A stimulation setting may include an electrode configuration and values for one or more stimulation parameters (P₁, P₂, . . . , Pₘ) 645. The electrode configuration includes information about electrodes (ring electrodes and/or segmented electrodes) selected to be active for delivering stimulation (ON) or inactive (OFF), polarity of the selected electrodes, electrode locations (also referred to as contact locations, which may include longitudinal positions of ring electrodes along the length of a lead, or angular positions of segmented electrodes about a circumference of a cross-section of the lead at a longitudinal position), and stimulation modes (e.g., monopolar pacing or bipolar pacing), etc. The stimulation parameters may include, for example, current amplitude values, current fractionalization across electrodes, stimulation frequency, stimulation pulse width, etc. In some examples, the feedback control logic 641 may modify the stimulation setting 644 such as by changing a stimulation parameter value or modifying an electrode configuration.

The stimulation setting 644 may be provided to the DBS controller 642 to configure the IPG or ETM to deliver DBS therapy to the patient 646 in accordance with the stimulation setting or the modified stimulation setting. The stimulation may produce certain therapeutic effects and/or side effects on the patient. Such therapeutic effectiveness and side effects, also referred to as clinical responses or clinical metrics, may be provided to the feedback control logic 641. In an example, the clinical responses may be based on patient or clinician observations. For example, motor symptoms such as bradykinesia (slowness of movement), rigidity, tremor, among other symptoms or side effects, may be scored by the patient or by the clinician upon overserving or questioning the patient. In some examples, the clinical responses may be objective in nature, such as measurements automatically or semi-automatically taken by a sensor 647. In an example, the sensor 647 may be included in a wearable device associated with patient 646, such as a smart watch. For example, a Parkinson's patient may be fitted with a wearable sensor that measures tremors, such as by measuring the frequency and amplitude of such tremors.

The clinical responses, either reported by the patient or measured by a sensor, may be converted to clinical response values 648, also referred to as clinical response scores. In an example, the clinical response values 648 may be computed based on the intensity, frequency, or duration of one or more of tremor, rigidity, or bradykinesia responses. Based upon the received clinical response values 648, the feedback control logic 641 may adjust electrode configurations or values of one or more stimulation parameters 645. The feedback control logic 641 may send the adjusted (new or revised) stimulation setting 644, such as the electrode configuration or the adjusted stimulation parameter values, to further configure the DBS controller 642 to change the stimulation parameters of the leads implanted in patient 646 to the adjusted values.

The feedback-control loop may continue until an optimal, desired, or acceptable outcome is reached, such as maximizing therapeutic effectiveness while minimizing unwanted side effects, or until a specific stop condition is reached such as number of iterations, time spent in programming session, or the like. An outcome may be considered optimal, desired, or acceptable if it meets certain threshold values or tests (e.g., improved clinical response for the patient, faster programming of the device, increased battery life, and/or control multiple independent current sources and directional lead). Such an iterative process of looking for a stimulation setting (e.g., an electrode configuration and stimulation parameter values for the electrode) is referred to as a stimulation setting optimization process. The outcome being reached may be referred to as an optimization criterion, and the resultant stimulation setting may be referred to as an optimal base stimulation setting (BSS). By way of example and not limitation, the optimization criterion may include possible optimal clinical outcome within the parameters chosen; time spent, iterations taken, or power usage to explore the search space until a desired clinical outcome is reached (assuming multiple outcomes with the same or comparable clinical response); among others.

In an example, the optimization criterion includes the clinical response values exceeding a threshold value or falling into a specified value range, indicating a satisfactory therapeutic outcome has reached. Depending on how the clinical response values are computed, one or more optimal base stimulation settings may be determined. For example, the clinical response values may be computed using a single response effect (e.g., one of bradykinesia, tremor, or rigidity). The optimal base stimulation settings (e.g., BSS₁ through BSS₄), may be stored in the memory 649. In an example, a stimulation setting, along with the corresponding unique clinical response indicator (e.g., weighted combination of clinical effects with unique weight factors) form a stimulation program 650, which may also be stored in the memory 649. Each stimulation program may be associated with, or tagged by, one or more unique clinical response indicators. In some examples, the clinical response values 648 may be weighted according to the time at which the test took place.

In various examples, the stimulation parameter control system 640 may be executed on its own and is not connected to a controller. In such instances it may be used to merely determine and suggest programming parameters, visualize a parameter space, test potential parameters, etc.

The process of searching for a stimulation setting (e.g., an electrode configuration and/or stimulation parameter values) typically involves significant computation and time, especially when electrode configuration involves segmented electrodes in a directional lead. If testing all possible settings in the entire parameter space (including electrode configurations and combinations of stimulation parameter values) is done as comprehensively as possible, stimulation would need to be provided to the patient for each possible setting, which may end up with a burdensome and time-consuming programming session. Because practically a programming session may only last a few hours, only a fraction of possible electrode configuration and stimulation parameter combinations may reasonably be tested and evaluated. To reduce the time taken and to improve the efficiency of stimulation setting optimization process, a reduced or restricted electrode configuration and parameter search space may be used. By applying limitations or constraints to the electrode configurations and parameter values, the restricted search space may include a subset of electrodes (e.g., a subset of ring electrodes and/or a subset of segmented electrodes on a lead) that are selected as active electrodes for delivering stimulation, and values or value ranges for one or more stimulation parameters (e.g., a range of current amplitude ranges for an active electrode). Stimulation setting optimization, when performed within such a search space, may be more efficient and cost-effective than searching through the entire parameter space for one or more optimal base stimulation settings.

The search space identifier 643 may automatically determine a search space 651 for a stimulation lead at a neural target, such as a region in a brain hemisphere, by imposing certain limitations or constraints on the electrode configurations and/or parameter values or value ranges. In an example, the search space 651 may be determined based on spatial information of the lead, such as lead positions with respect to neural targets, which may be obtained from imaging data of the lead and patient anatomy. Additionally, or alternatively, the search space 651 may be determined based on physiological information such as physiological signals sensed by the electrodes at their respective tissue contact locations. The physiological information may include patient clinical responses to stimulation. In some examples, prior knowledge about patient medical condition, health status, DBS treatment history may be used to determine the search space 651. In an example, the search space identifier 643 may exclude those electrodes on the lead that are out of a region of interest, such that the search space includes only those electrodes within the target of interest. One or more stimulation parameters may be restricted to take certain values or within value ranges. For example, the restricted search space may include certain electrode positions and value ranges for stimulation current amplitude, frequency, or pulse width. The feedback control logic 641 may determine one or more optimal base stimulation settings by searching through the identified search space 651. The identified search space 651 may be stored in the memory 649.

The feedback control logic 641 may include a machine learning engine 652 that may facilitate the stimulation parameter control system 640 (or a user of the system) to explore the search space in order to choose values for programming the DBS controller 642. The machine learning engine 652 may employ supervised or unsupervised learning algorithms to train a prediction model, and use the trained prediction model to predict patient clinical responses to an untested stimulation setting (e.g., untested stimulation parameter values or untested electrode configurations), or to estimate or predict stimulation parameters values or electrode configurations that, when provided to the DBS controller 642 to deliver stimulation accordingly to the patient 646, would produce desired or improved clinical responses. Examples of the learning algorithms include, for example, Naive Bayes classifiers, support vector machines (SVMs), ensemble classifiers, neural networks, Kalman filters, regression analyzers, etc. The machine learning engine 652 may build and train a prediction model using training data, such as stimulation parameter values and corresponding patient clinical responses. The training data may be acquired from a training session such as performed in a clinic. Additionally, or alternatively, the training data may be obtained from historical data acquired by the stimulation parameter control system 640. With its learning and prediction capability, the machine learning engine 652 may aid a user (e.g., a clinician) in exploring the stimulation parameter space more effectively and more efficiently to produce results that are optimal, desired, or acceptable.

In some examples, the machine learning engine 652 may use imaging data to inform the choice of the next set of values, which may be used when the algorithm finds itself in a region of parameter space for which the clinical responses are not substantially affected by the changes in the stimulation parameters, and the choice of next step is not apparent from the patient response alone. Imaging data that provides information about the location of the lead in the patient's brain along with priors informing the algorithm of which directions may be better choices for the next step could lead to faster convergence.

In some examples, the machine learning engine 652 may determine expected outcomes for parameter values that have not yet been tested based upon what the machine learning engine 652 has “learned” thus far and provide a recommendation for a next set of values to test. Here, testing refers to the iterative testing required to find an optimal stimulation setting for configuring the DBS controller 642. The recommendation for a next set of values to test is based upon which of the determined expected outcomes meet a set of designated (determined, selected, preselected, etc.) criteria (e.g., rules, heuristics, factors, and the like). For example, rules considered may include such factors as: the next set of values may not be one of the last 10 settings tested or may not be too close to previously tested setting. Accordingly, the feedback control logic 641 with its machine learning engine 652 is used to systematically explore the stimulation parameter space based upon what it has learned thus far and (optionally) different rules and/or heuristics that contribute to achieving optimal outcomes more efficiently.

The process for determining expected outcomes for parameter values that have not yet been tested may involve use of other data for machine learning. For example, data from other programming sessions for the same patient as well as from other patients may be used to train the machine learning engine 652. In some examples, no prior data may be used. In this case, the machine learning engine 652 may use data learned from this patient only in one particular setting. In other examples, data from the same patient but from previous sessions may be used. In some examples all patient data from all sessions may be used. In some examples all patient data utilizing lead location information (knowledge of lead location in space relative to anatomy) may be used. Different other combinations are also possible.

In order to use this data for machine learning purposes, the data may first be cleansed, optionally transformed, and then modeled. In some examples, new variables are derived, such as for use with directional leads, including central point of stimulation, maximum radius, spread of stimulation field, or the like. Data cleansing and transformation techniques such as missing data imputation and dimension reduction may be employed to prepare the data for modeling.

The machine learning engine 652 may determine how best a predicted outcome meets the optimal outcome metrics. Various optimization techniques may be used, examples of which may include but are not limited to: optimization algorithms and estimation procedures used to fit the model to the data (e.g., gradient descent, Kalman filter, Markov chain, Monte Carlo, and the like); optimization algorithms reformulated for search (e.g., simulated annealing); spatial interpolation (e.g., kriging, inverse distance weighting, natural neighbor, etc.); supplementary methods that aid the optimization process (e.g., variable selections, regularization, cross validation, etc.); other search algorithms (e.g., golden-section search, binary search, etc.). Using any of these techniques, the machine learning engine 652 may decide whether a particular predicted outcome for a set of stimulation parameter values is the fastest sufficing outcome, the best possible clinical outcome, or the optimal outcome with least battery usage, for example.

The feedback control logic 641 may be used to search and configure different types of stimulation parameters of the various leads potentially causing different clinical effects upon the patient. Examples of the stimulation parameters may include electrode configurations (electrode selection, polarities, monopolar or bipolar modes of stimulation), current fractionalization, current amplitude, pulse width, frequency, among others. Given these possible stimulation parameters, the stimulation parameter control system 640 may move about the parameter space in different orders, by different increments, and limited to specific ranges. In some examples, the stimulation parameter control system 640 may allow the user to provide search range limitations to one or more of the stimulation parameters to limit the range for that stimulation parameter over which the system will search for parameters. For example, the user may restrict which electrodes may be used for stimulation or may restrict the amplitude or pulse width to a certain range or with a selected maximum or minimum. As one illustration, based on the site of implantation, the user may be aware that the distal-most and proximal-most electrodes are unlikely to produce suitable stimulation and the user limits the range of electrodes to exclude these two electrodes.

For a lead with segmented electrodes, the number of possibilities for parameter selection may be very large when combinations of electrodes and different amplitudes on each electrode are possible. In some examples using a lead with segmented electrodes, the selection of electrodes used for stimulation may be limited to fully directional selections (i.e., selection of only a single segmented electrode) and fully concentric selections (i.e., all electrodes in a single set of segmented electrodes are active with the same amplitude). In other examples, the initial movement through parameter space may be limited to fully directional and fully concentric selections. After a set of stimulation parameters is identified using these limits, variation in the selection of electrodes may be opened up to other possibilities near the selection in the identified set of stimulation parameters to further optimize the stimulation parameters.

In some examples, the number of stimulation parameters that are varied and the range of those variations may be limited. For example, some stimulation parameters (e.g., electrode selection, amplitude, and pulse width) may have larger effects when varied than other stimulation parameters (e.g., pulse shape or pulse duration). The movement through stimulation parameter space may be limited to those stimulation parameters which exhibit larger effects. In some examples, as the stimulation parameter control system 531 proceeds through testing of sets of stimulation parameters, the system may observe which stimulation parameters provide larger effects when varied and focus on exploring variation in those stimulation parameters.

In some examples, the stimulation parameter control system 640 may include a user interface for visualizing exploration of the stimulation parameter space as the system determines new and better parameter values to test until a solution is determined that fits within certain designated thresholds or a stop condition is reached. In some examples of the stimulation parameter control system 640, the user interface is part of the feedback control logic. In other examples, the user interface may be part of another computing system that is part of the stimulation parameter control system 640 or may be remote and communicatively connected to the stimulation parameter control system 640. The user interface may present to a user (such as a clinician, physician, programmer, etc.) a visualization of the predicted expected outcomes for (some of) the stimulation parameter values not yet tested and a recommendation for the next set of stimulation parameter values to test.

In some examples where a deep brain stimulator is configured via the DBS controller 642 with at least one set of stimulation parameter values forwarded by the feedback control logic 641, the clinician may monitor the patient throughout the process and record clinical observables in addition to the patient 646 being able to report side effects. When a side effect is observed, the various search algorithms may take that fact into account when selecting/suggesting a next set of values to test. In some examples, for example, those that select contacts via monopolar review, other parameters may be changed until they cause a side effect, which case is noted as a boundary. For example, in monopolar review where amplitude is another stimulation parameter being varied, the amplitude may be increased progressively (e.g., via a variable ramp protocol) until a side-effect is observed.

In some examples, more than one clinical metric (e.g., tremor, rigidity, bradykinesia, etc.) may be important observables. Different examples of the stimulation parameter control system 640 may handle these metrics differently. For example, some examples might identify an ideal location for each metric and choose one ideal location between them, set in the patient's remote controller so the patient may choose as needed, or chose a best combined outcome. As another example, some examples may search multiple outcomes at the same time and use the best combined score as the best outcome or find a best location for each metric individually. As yet another example, some examples may use a sequential process for selecting stimulation parameter values for multiple outcomes. For example, a system may search parameter space for a first outcome (e.g., bradykinesia) and, upon finding a suitable end condition, then search parameter space for a second outcome (e.g., rigidity). While searching parameter space for the first outcome, clinical response values for both the first and second outcomes may be obtained. Thus, when the system switches to the second outcome there are already a number of clinical response values for that outcome which will likely reduce the length of the search.

In some examples, two stimulation leads may be implanted to produce stimulation effects on two sides of the body (e.g., the right and left sides of the body). The same procedure described herein may be used to either jointly determine the stimulation parameters for the two leads by exploring the joint parameter space or individually determine stimulation parameters for the two leads by exploring the parameter space for each lead individually. In some examples, the user may determine for each side of the body which clinical response is dominant or most responsive. This may be done, for example, by having the patient perform a single task which captures multiple responses (e.g., connecting dots on the screen to monitor tremor and bradykinesia of the movement) or a small series of tasks. This enables the system to determine which clinical response to use to identify the stimulation parameters for that side of the body.

As noted, the feedback may be provided directly by the patient 646, entered by an observer such as a clinician (not shown), or may be provided by means of a sensor 647 associated with and in physical, auditory, or visual contact with the patient 646. Examples may include, but are not limited to, accelerometers, microphones, and cameras. In an example, the sensor 647 may be included in a wearable device associated with patient 646, such as a smart watch. In an example where the feedback may be monitored automatically or semi-automatically, such as with use of sensor 647, it may not be necessary for a clinician or other observer to be present to operate the stimulation parameter control system 640. Accordingly, in such examples a user interface may not be present in system 640.

In some examples, the stimulation parameter control system 640 may determine one or more optimal base stimulation settings using predicted clinical responses for untested stimulation parameter values or untested electrode configurations without actually delivering stimulation. Such base stimulation settings are referred to as estimated or predicted base stimulation settings, to distinguish from the tested base stimulation settings that are based on the tested clinical response (either reported by the patient or measured by a sensor) to actually delivered stimulation. For examples, based on the “tested” base stimulation settings, the stimulation parameter control system 640 may estimate an optimal base stimulation setting associated with a composite clinical response defined as x%*bradykinesia+y%*tremor+z%*rigidity, or simply denoted by the weight factors (x%, y%, z%). In an example, the stimulation programs 650 may be stored in a lookup table, where each tested or estimated base stimulation setting (e.g., BSS₁ through BSS₈) may be tagged by respective clinical response indicators or weight factors for clinical effects. In an example, the memory 649 may be a part of memory circuitry internal to the IPG. The RC or the CP may request access to the memory 649 to retrieve therefrom one or more stored stimulation programs 650 or the search space 651.

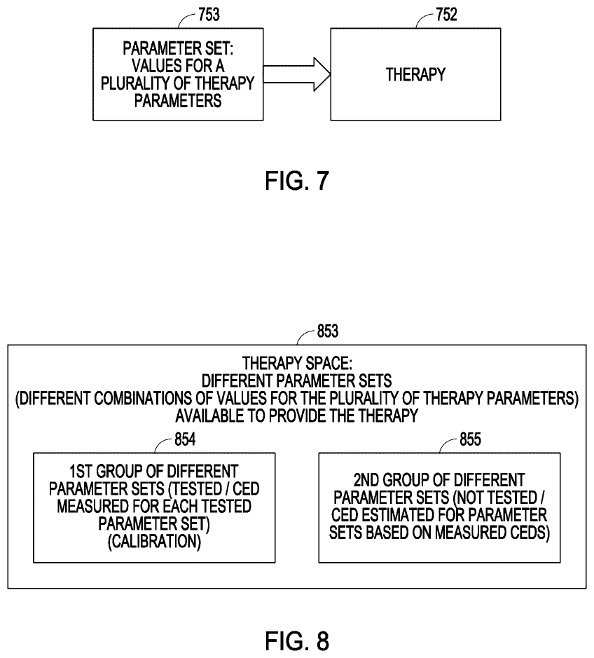

FIG. 7 illustrates a therapy 752 being delivered according to a parameter set. The parameter set may be programmed into the device to deliver the specific therapy using specific values for a plurality of therapy parameters. By way of example and not limitation, the therapy parameters that control the therapy may include pulse amplitude, pulse frequency, pulse width, and electrode configuration (e.g., selected electrodes, polarity and fractionalization). The parameter set includes specific values for the therapy parameters.

FIG. 8 illustrates a therapy space, which includes different parameter sets potentially available for delivering the therapy. The different parameter sets 853 have unique combinations of values for the therapy parameters. The therapy space may be burdensomely large as there may be many unique combinations of values for therapy parameters (e.g., many unique parameter sets). Some parameter sets within the therapy space may be tested and the corresponding clinical effect data (CED) may be measured or otherwise acquired for the tested parameter sets. These tested parameter sets are illustrated as a first group of different parameter sets within the therapy space 854. Other parameter sets may not be tested (e.g., second group of different parameter sets 855). The CED for these parameter sets may be estimated based on measured CEDs for the patient or a patient population.

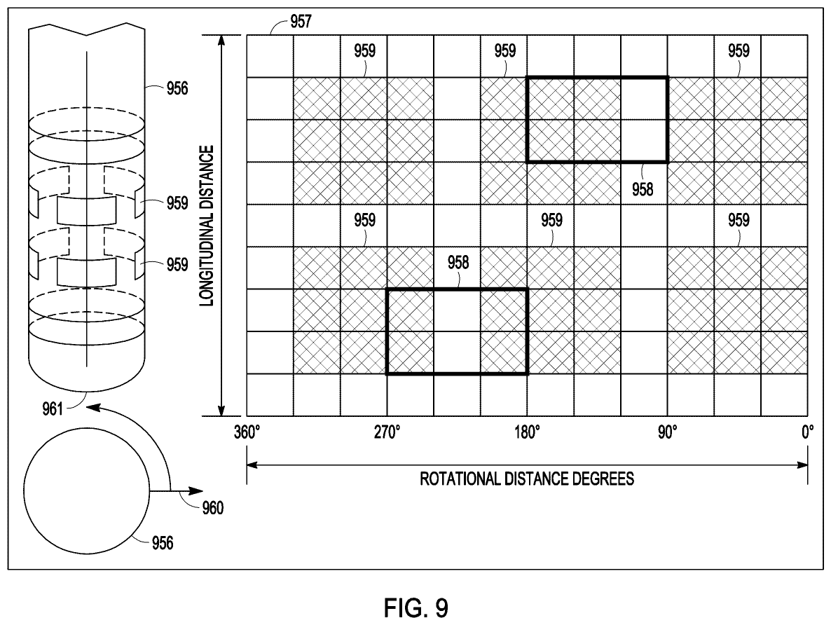

FIG. 9 illustrates, by way of example and not limitation, an example of a directional lead 956 and a representation of a grid 957 used to set up a virtual electrode layout, which defines a virtual electrode's (VE's) voltage field. A best fit voltage field generated by the physical electrodes 959 on the directional lead 956 may represent the VE's voltage field for a particular steering state. It is noted that the VE position is not limited by the grid used to set up the voltage field and that the grid is not necessarily set up to the physical electrodes on the lead. Different sets of electrodes may be active and used to program the neuromodulation to stimulate target(s). The target(s) may be identified using existing anatomical structures, hotspot searching using sensor feedback as a predictive marker for patient outcomes (e.g., symptom relief), or a heat map developed based on effects experienced by the patient. The stimulation may be monopolar or bipolar. In a monopolar stimulation, for example, all of the active electrodes may contribute a polarity of the delivered energy. If cathodic neuromodulation is delivered using monopolar stimulation, then all of the active electrodes contribute toward the total cathodic current delivered by the neuromodulation. In a bipolar stimulation, for example, some of the active electrodes may be cathodic active electrodes and other active electrodes may be anodic active electrodes at one point of time. The cathodic active electrodes contribute toward the total cathodic current delivered by the neurostimulation and the anodic active electrodes contribute toward the total anodic current delivered by the neurostimulation.

A normal vector is a representation of the steering state of directional stimulation on a directional lead. For linear leads and directional leads that are configured to stimulate in a ring (or full row), the steering state may be given by a single value representing the location of the stimulation along the lead. VEs may be created to model electrodes that are less than a full ring electrode but are larger than a segmented electrode on a directional lead. The size of these VEs may be referred to as “spread” or “focus.” A vector may extend from a VE to provide a representation of a stimulation field (e.g., SFM). For a given amplitude, the SFM for a VE with a relatively larger spread is wider and flatter than a VE with a relatively smaller spread. Current programming algorithms may control a position and magnitude of a vector 960, such as the illustrated vector extending in a normal direction away from the lead. The position of the virtual electrode 958 along the directional lead 956 may be used to identify the origin of the vector. The position and size of the virtual electrode 958 is controlled by the fractionalized contributions of the physical electrodes 959. The directional lead 956 includes a distal end 961 and the virtual electrode 958 may be at different distances from the distal end and also may be at different rotational positions around the directional lead 956. For example, the total cathodic current contributions may be equally divided over two active physical electrodes in the same row such that each electrode as 50% of the total cathodic current. The resulting virtual electrode would be centered between the two active physical electrodes. The magnitude of the vector 960 is controlled by the amount of energy applied to the electrodes. For example, models have been developed to illustrate the stimulation field (e.g., stimulation field model) for a stimulation parameter set (e.g., current amplitude, pulse width, and rate). The grid 957 may be used to define the shape and weight of various subcomponents of the virtual electrode that is used to generate a voltage field. A linear least squared inverse fitting function, using the VE voltage field, voltage fields from physical electrodes, and a steering state, generates a bipolar fractionalization for that steering state, that real lead and that VE. The grid does not represent an overlay on the electrodes of the physical leads as it is used to only generate a voltage field for the VE.

Various embodiments of the present subject matter define a position and magnitude of two or more vectors. Different vectors may be in the same row (e.g., same distance from the distal end but at different rotational positions). Different vectors may be at the same rotational direction but different distances from the distal end 961. As illustrated in the grid, virtual electrodes 958 may be both at different distances from the distal end and may be at different rotational positions around the directional lead.

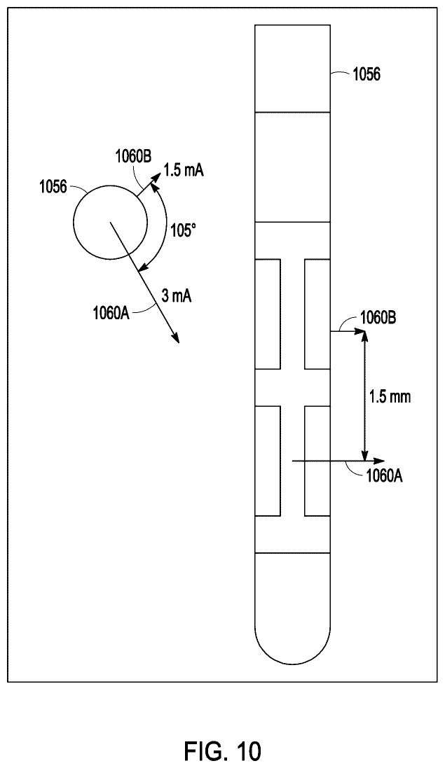

FIG. 10 illustrates, by way of example and not limitation, two vectors 1060A and 1060B extending away from a directional lead 1056. The vectors illustrated on the cross section of the lead shows that the first vector 1060A has a magnitude of 3 mA and the second vector 1060B has a magnitude of 1.5 mA. The rotational angle between the two vectors 1060A and 1060B is 105°. The vectors are found to modulate targets. For each vector, fractionalizations may be calculated to create the vector. Testing may be performed as part of the process to find the desired targets and the vector(s) may be adjusted accordingly. The fractionalizations for more than one target may be combined into a combined modulation configuration in a manner to preserve the original target stimulation. The combined modulation configuration may be used to deliver neuromodulation via a single timing channel to modulate the desired targets.

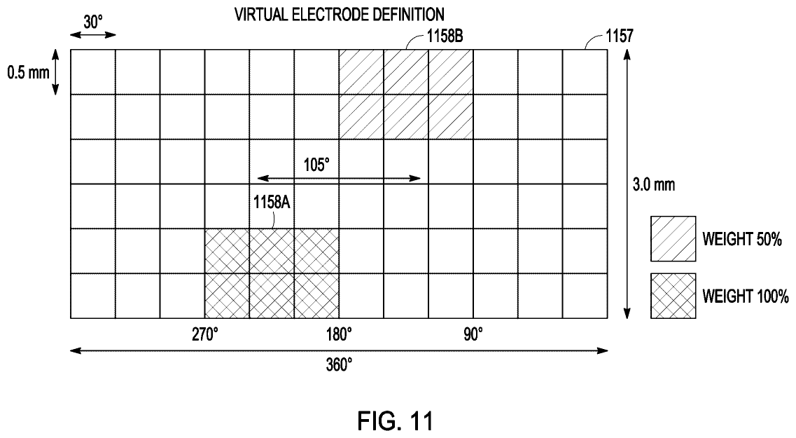

FIG. 11 illustrates, by way of example and not limitation, a virtual electrode steering grid 1157 for the vectors illustrated in FIG. 10. The steering grid 1157 may represent a virtual electrode in a first amplitude and steering state 1158A that corresponds to vector 1060A in FIG. 10, and in a second amplitude and steering state 1158B that corresponds to vector 1060B in FIG. 10. Also, the magnitude of the illustrated virtual electrode in the first steering state 1158A is 100% of the amplitude (e.g., 3 mA) and the magnitude of the virtual electrode in the second steering state 1158B is 50% of the amplitude (e.g., 1.5 mA). As the lower virtual electrode is weighted at 100% and the upper virtual electrode is weighted at 50%, the lower portion of the field extends further away from the lead than the upper portion of the field.

Current programming tools create a stimulation field model (SFM). that may be steered during a programming process to cover a desired region and not modulate an avoidance region. The model for the stimulation field is based on a stimulation parameter set that includes a fractionalization. The model may also be based on other parameters in the set such as pulse width and frequency.

The current programming tools independently determine solutions (e.g., fractionalizations) to stimulate different targets, and different timing channels may be used to stimulate the different targets at different times. Different stimulation channels (also referred to as areas) are not able to stimulate exactly at the same time as they may be designed with arbitration to avoid undesired interactions between the active electrodes. For example, if arbitration was not used, the cumulative amplitude may be larger than expected. However, timing is very important in neuroscience, and the use of different timing channels for the independently-determined solutions to stimulate the different targets undesirably introduce an element of time. The present subject matter is able to deliver the stimulation fields to simultaneously stimulate the different targets using the same timing channel. Electrophysical data (e.g., EP, LFP or other) may be used to activate two or more target areas by identifying two or more spatial maxima of the three-dimensional distribution. An inverse calculation of the fractionalization of active physical electrodes may be determined. Some embodiments may avoid activation of a region of the lead that points to the minima (i.e., where stimulation is not desired). Stimulation may be refined using sensor(s) to iteratively optimize workflow and consideration of concomitant or interleaved pulse patterns. Stimulation may further be refined using clinical responses from the patient.

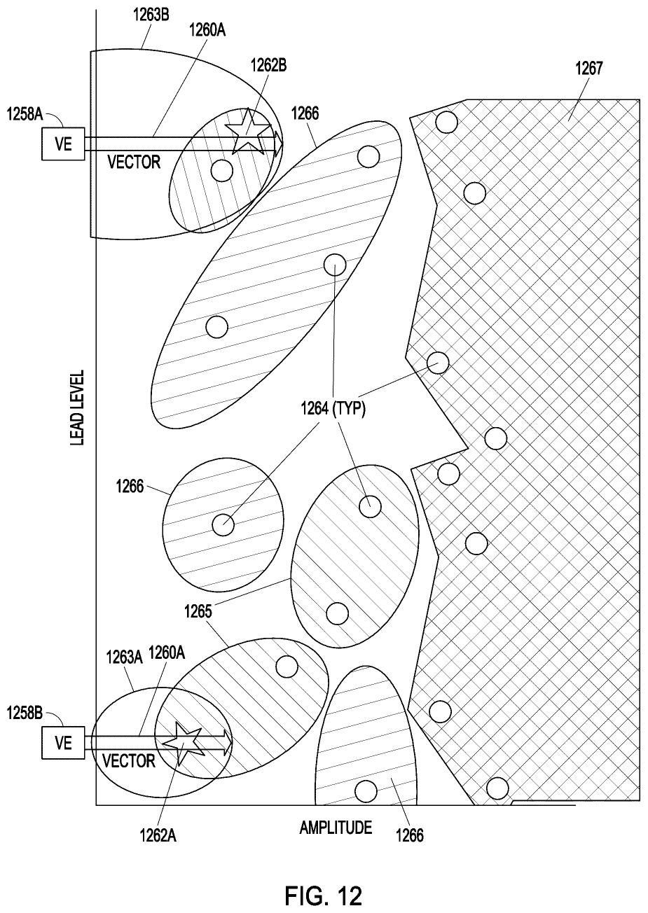

FIG. 12 illustrates, by way of example and not limitation, a map illustrating good regions for modulation, side effect regions, and regions that have some benefit and no side effects, and also illustrating stimulation field models (SFMs) for two vectors from two virtual electrodes (VEs). The vertical axis represents the lead level (e.g., longitudinal distance along the lead). Two virtual electrodes (VEs) 1258A and 1258B are illustrated along the lead level. The horizontal axis represents amplitudes of vectors 1260A and 1260B. Stars 1262A and 1262B represent the modulation targets. The map also includes circles 1264 representing tested parameter sets within a search space. The tested regions are used to collect data used to estimate regions 1265 where modulation is desirable, regions 1266 where modulation has some benefit and does not cause a side effect(s), and regions 1267 where modulation would cause a side effect. By way of example, a first target may be therapeutic (e.g., address the patient's tremor rigidity) and a second target may counteract aside effect (e.g., counteract potential dyskinesia that is emerging the patients. In an example, one target may help tremor or speech or excessive movements. In an example, modulation of one target may help with general Parkinson's, but because gait does not usually respond in the same place, it may be possible to hit a center that is better with gait.

Previously, separate timing channels were used to provide a modulation field (e.g., stimulation field model (SFM) 1263A and 1263B) over the modulation targets, where one target is hit using a vector in one timing channel and the other target is hit by another vector in another timing channel. In contrast, various embodiments of the present subject matter are able to automatically target both of these regions on the same timing channel.

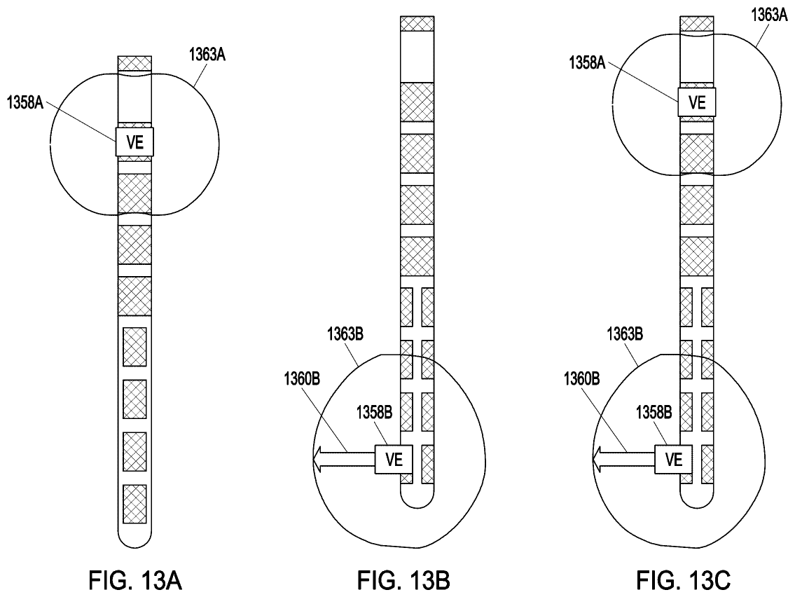

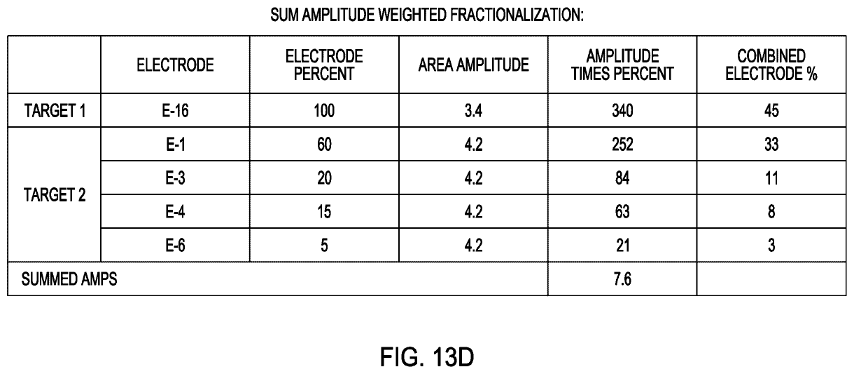

FIGS. 13A-13D illustrate, by way of example, a first configuration to modulate a first target, a second configuration for to modulate a second target, and a combination configuration to modulate both the first target and the second target. The first stimulation configuration corresponds to the first SFM 1363A using a first virtual electrode (VE) 1358A, as illustrated in FIG. 13A. The first VE 1358A is a ring electrode. The second stimulation configuration corresponds to the second SFM 1363B as defined by a second vector 1360 extending from a second virtual electrode (VE) 1363B, as illustrated in FIG. 13B. Currently, using known programming tools, the first SFM 1363A shown in FIG. 13A may be provided using a first timing channel (area) and the second SFM 1363B shown in FIG. 13B may be provided using a second timing channel (area). The stimulation amplitude in the first timing channel may be delivered using 3.4 mA (area amplitude) where 100% of the energy (e.g., cathodic energy) may be delivered using electrode E-16. For example, the stimulation amplitude in the second timing channel may be 4.2 mA distributed over several electrodes (e.g., fractionalization of 60% of the energy delivered using electrode E-1, 20% of the energy delivered using electrode E3, 15% of the energy delivered using electrode E-4, and 5% of the energy delivered using electrode E-6.

Various embodiments of the present subject matter are able to automatically target both of these regions on the same timing channel. FIG. 13C illustrates both the first SFM 1363A and second SFM 1363B delivered at the same time using the same timing channel, and FIG. 13D illustrates the table that illustrates combining the first modulation configuration and the second modulation configuration into a combined modulation configuration. By way example and not limitation, one embodiment of combining two SFM targets is to amplitude-weight the fractionalizations and divide by the summed amplitude and set the area amplitude to the combined amplitude. For each active electrode in a configuration, the area amplitude is multiplied by a percentage that represents the corresponding fractionalized contribution. The sum of the first area amplitude (e.g., 3.4 mA) and the second area amplitude (e.g., 4.2 mA) provide the total area amplitude (7.6 mA) for the combination configuration. Thus, for example, the product of 100 and 3.4 is 340 for electrode E-16, the product of 60 and 4.2 is 252 for electrode E-1, the product of 20 and 4.2 is 84 for electrode E-3, the product of 15 and 4.2 is 63 for electrode E-4, and the product of 5 and 432 is 21 for electrode E-6. These product values are divided by the summed amplitude 7.6 to provide the fractionalized contribution of each active electrode to the overall summed amplitude (7.6) for the timing channel. Thus, the fractional value for electrode E-16 is 45%, the fractional value for electrode E-1 is 33%, the fractional value for electrode E-3 is 11%, the fractional value for electrode E-4 is 8% and the fractional value for electrode E-6 is 3%. A 7.6 mA current in one timing channel fractionalized across E-1, E3, E-4 and E-6 will thus approximate the SFM 1363A in FIG. 13A delivered using a current amplitude of 3.4 mA delivered in a first channel to one electrode and the SFM 1363B in FIG. 13B delivered using a current amplitude of 4.2 mA delivered in a second channel distributed among four electrodes.

The system may be configured to automatically perform these calculations in response to distinctly programming a first vector 1360A from a first VE 1358A in FIG. 13A to modulate a first target and programming a second vector 1360B from a second VE 1358B in FIG. 13B to modulate a second target. For example, the system may include software and/or firmware instructions to perform the calculations. There may be a number of processes to determine the combination configuration. For example, the processes may include combining fractionalizations and amplitudes, matching voltage fields, or iteratively matching SFMS and current tables (Ith tables) with the corresponding fractionalizations to create those SFMs.

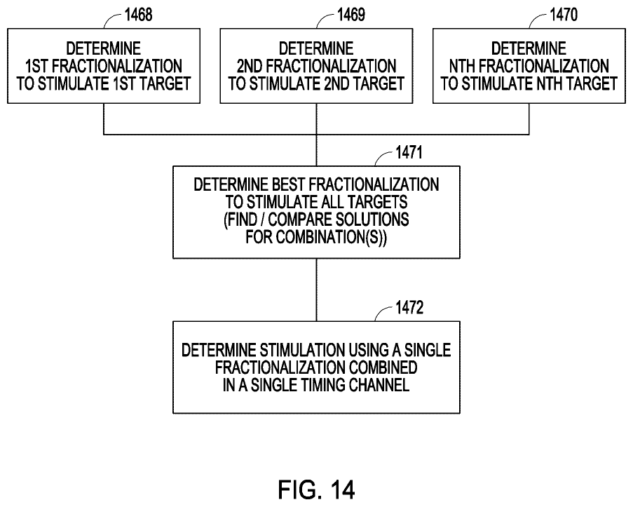

FIG. 14 illustrates, by way of example and not limitation, a method for creating a modulation configuration to stimulate two or more targets. The method includes determining fractionalization(s) (e.g., solution(s)) to stimulate target(s). For example, the method may include determining a first fractionalization to stimulate a first target at 1468, determining a second fractionalization to stimulate a second target at 1469, and determine an Nth fractionalization to stimulate an Nth target at 1470. Each of these N fractionalizations may be referred to as a steering state. At 1471, a best fractionalization to stimulate all targets may be determined. For example, solutions for different combinations of steering states (e.g., fractionalizations) may be determined and then compared to find the best solution. For example, instead of determining the fractionalization for a single steering state/VE at a time, the fractionalizations for two steering states and two VEs may be determined at a time and the best solution may be determined. In a specific example, (1) Ring 1 and Ring 2 may be optimized to get best solution A, (2) Ring 1 and Directional 2 may be optimized to get best solution B, (3) Directional 1 and Ring 2 may be optimized to get best solution C, (4) Directional 1 and Directional 2 may be optimized to get best solution D, and (5) Use best solution across A B C and D. At 1472, the stimulation may be delivered using a single fractionalization combined in a single timing channel. The final solution (e.g., combined fractionalization for a single timing channel) may be steered. Thus, for example, the fields used to stimulate the targets may be moved together.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media (referred to herein as computer readable medium), such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine” may include at least one processor/controller, including one processor/controller to implement all of the instructions, at least two processors/controllers where one processor/controller operates on some of the instructions and other processor(s)/controller(s) operate on other instructions, or at least two processors/controllers where each processor/controller is capable of operating on the same instructions. Thus, for example, distributed systems or systems with shared resources are contemplated.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
determining a first fractionalization to stimulate a first target using a neurostimulator that includes a plurality of electrodes;
determining a second fractionalization to stimulate a second target using the neurostimulator;
determining a combined fractionalization, using the first fractionalization and the second fractionalization, for stimulating both the first target and the second target using a single timing channel in the neurostimulator; and
stimulating the first target and the second target using the single timing channel in the neurostimulator and the combined fractionalization.

2. The method of claim 1, wherein the first fractionalization is part of a first stimulation parameter set configured for use by the neurostimulator to stimulate the first target, the second fractionalization is part of a second stimulation parameter set configured for use by the neurostimulator to stimulate the second target, the first stimulation parameter set and the second stimulation parameter set have a same pulse width and a same frequency, and the first target and the second target are simultaneously stimulated using a combined parameter set that includes the combined fractionalization, the same pulse width and the same frequency.

3. The method of claim 1, the combined fractionalization is determined by weighting the first fractionalization and the second fractionalization.

4. The method of claim 3, further comprising:
determining a first area amplitude for stimulating the first target and distributing the first area amplitude among a first set of active electrodes according to the first fractionalization;
determining a second area amplitude for stimulating the second target and distributing the second area amplitude among a second set of active electrodes according to the second fractionalization; and
determining a combined amplitude by summing the first area amplitude and the second area amplitude,
wherein the combined fractionalization is determined by amplitude weighting a fractional contribution for the first set of active electrodes using the first area amplitude and dividing by the combined amplitude and by amplitude weighting a fractional contribution for the second set of active electrodes using the second area amplitude and dividing by the combined amplitude; and
the combined amplitude and the combined fractionalization are used to stimulate the first target and the second target using the single timing channel in the neurostimulator.

5. The method of claim 1, wherein the neurostimulator includes at least one lead, the plurality of electrodes are on the at least one lead, and the at least one lead includes a directional lead or a linear lead.

6. The method of claim 1, wherein stimulation of the first target addresses a first clinical benefit or side effect and stimulation of the second target addresses a second clinical benefit or side effect.

7. The method of claim 1, further comprising using a programming tool to both determine the first fractionalization to stimulate the first target and determine the second fractionalization to stimulate the second target.

8. The method of claim 7, further comprising using the programming tool to determine neurostimulation target information indicative of at least one of the first target and the second target by:
receiving the neurostimulation target information via a user interface;
receiving medical imaging data;
receiving a sensor signal indicative of a sensed parameter; or
receiving user inputs regarding at least one a clinical effect or side effect of the neurostimulation.

9. The method of claim 7, further comprising using the programming tool to determine neurostimulation target information indicative of at least one of the first target and the second target using at least one of:
anatomical information indicative of an anatomical structure;
electrophysical data; or
neuromodulation response information, wherein the neuromodulation response information includes at least one of a heat map indicative of a desired response and/or undesired response to neuromodulation sites, sensor feedback when neuromodulation is delivered at neuromodulation sites, or user feedback indicative of symptom relief and/or experienced side effects when the neuromodulation is delivered at the neuromodulation sites.

10. The method of claim 1, further comprising determining a third fractionalization to stimulate a third target, wherein the combined fractionalization is determined to simultaneously stimulate the first target, the second target and the third target using the single timing channel, the combined fractionalization is determined using the first fractionalization, the second fractionalization, and the third fractionalization.

11. The method of claim 10, further comprising:
combining fractionalizations using different combinations of the first fractionalization, the second fractionalization and the third fractionalization to create a plurality of combined solutions; and
comparing the combined solutions to determine best fractionalization to stimulate all targets.

12. The method of claim 1, further comprising:
determining a first stimulation field to stimulate the first target;
determining the first fractionalization based on the first stimulation field;
determining a second stimulation field to stimulate the second target; and
determining the second fractionalization based on the second stimulation field.

13. The method of claim 1, further comprising receiving a first target input indicative of the first target and a second target input indicative of the second target.

14. The method of claim 13, further comprising receiving a first avoidance input indicative of a first avoidance region and a second avoidance input indicative of a second avoidance region.

15. The method of claim 1, wherein a first stimulation field corresponds to a first stimulation field model (SFM) that is based on the first fractionalization, and a second stimulation field corresponds to a second stimulation field model (SFM) that is based on the second fractionalization.

16. A non-transitory machine-readable medium including instructions, which when executed by a machine, cause the machine to perform a method, comprising:
determining a first fractionalization to stimulate a first target using a neurostimulator that includes a plurality of electrodes;
determining a second fractionalization to stimulate a second target using the neurostimulator;
determining a combined fractionalization, using the first fractionalization and the second fractionalization, for stimulating both the first target and the second target using a single timing channel in the neurostimulator; and
stimulating the first target and the second target using the single timing channel and the combined fractionalization.

17. A system, comprising:
a neurostimulator including a plurality of electrodes;
a processing system configured to perform a process that includes:
determining a first fractionalization to stimulate a first target using the neurostimulator;
determining a second fractionalization to stimulate a second target using the neurostimulator; and
determining a combined fractionalization, using the first fractionalization and the second fractionalization, for stimulating both the first target and the second target using a single timing channel in the neurostimulator; and
wherein the neurostimulator is configured to stimulate the first target and the second target using the single timing channel and the combined fractionalization.

18. The system of claim 17, wherein:
the first fractionalization is part of a first stimulation parameter set configured for use by the neurostimulator to stimulate the first target;
the second fractionalization is part of a second stimulation parameter set configured for use by the neurostimulator to stimulate the second target;
the first stimulation parameter set and the second stimulation parameter set have a same pulse width and a same frequency; and
a combined parameter set configured for use by the neurostimulator to simultaneously stimulate both the first target and the second target include the combined fractionalization, the same pulse width and the same frequency.

19. The system of claim 17, wherein the processing system includes a programming tool configured to both determine the first fractionalization to stimulate the first target and determine the second fractionalization to stimulate the second target.

20. The system of claim 17, wherein:
a first stimulation field corresponds to a first stimulation field model (SFM) that is based on the first fractionalization; and
a second stimulation field corresponds to a second stimulation field model (SFM) that is based on the second fractionalization.
